(19) United States
(12) Patent Application Publication
(10) Pub. No.: US 2012/0284907 A1
(43) Pub. Date: Nov. 15, 2012

(54) Goggle Structure

(75) Inventor: Chih-Cheng Shiue, Taipei (TW)

(73) Assignee: QBAS CO., LTD., Taipei (TW)

(21) Appl. No.: 13/336,988

(22) Filed: Dec. 23, 2011

(30) Foreign Application Priority Data
May 11, 2011 (TW) 100208449

(51) Int. Cl. A61F 9/02

(52) U.S. Cl. 2/431

(57) ABSTRACT

A goggle structure is disclosed, which includes: a facial mask, having a skirt portion and a nose portion connected with each other; a frame, connected to the skirt portion; a lens assembly, disposed within the frame; and a soft supporting member softer than the frame, disposed between the frame and the skirt portion, and at least located above the nose portion and extending toward the skirt portion. In this way, the soft supporting member can separate at least part of the frame from the skirt portion, resulting in that the frame will not be pressed against the user's face when an external force is exerted.

### CROSS-REFERENCES TO RELATED APPLICATIONS

This application claims priority to Taiwan Patent Application No. 100208449 filed on May 11, 2011, which is hereby incorporated herein by reference entirely.

### BACKGROUND OF THE INVENTION

1. Field of the Invention

The present invention relates to a goggle structure, and more particularly, to a goggle structure for use in water or underwater sports.

2. Descriptions of the Related Art

When participating in water or underwater sports, people usually wear goggles to prevent their eyes from coming into contact with water or other foreign matters. In reference to FIGS. 1 and 2, a conventional goggle structure 9 typically comprises a facial mask 91 and a frame 92. The frame 92 is disposed on the front side of the facial mask 91. When the goggle structure 9 is worn by a user, the facial mask 91 makes close contact with the user's face; and the frame 92 maintains a distance from the user's face without making contact with the user's face.

However, because the bandeau of the goggle structure is elastic, some users often unintentionally apply a large force to tighten the bandeau when wearing the goggle structure 9; or they may often subconsciously press and laterally push the goggle structure toward the face in expectation of a water-proof effect through a suction-like attachment between the face and the goggle structure. However, these kinds of actions will cause the hard frame 92 to be pressed against the face, thus making the user feel uncomfortable.

Furthermore, if the supporting ability (i.e., the strength) of the facial mask 91 itself is inadequate, then it is very likely that the facial mask 91 can collapse with water pressure when a user wearing the goggle structure is participating in water or underwater sports, especially during a scuba diving. In this case, it becomes easier for the frame 92 to press against the user's face, and the discomfort felt by the user is much more serious than when the goggle structure is worn normally. This not only compromises the quality of the experience in the diving sport but also imposes an unpredictable threat to the user's safety in the water or underwater sport, such as scuba diving.

Accordingly, an urgent need exists in the art to provide a goggle structure capable of overcoming the aforesaid shortcomings.

### SUMMARY OF THE INVENTION

An objective of the present invention is to provide a goggle structure, which can overcome the problems of discomfort and threat to safety caused by the frame that comes into contact with the user's face.

To achieve the aforesaid objective, the goggle structure of the present invention comprises: a facial mask, having a skirt portion and a nose portion connected with the skirt portion; a frame, connected to the skirt portion; a lens assembly, disposed within the frame; and a soft supporting member softer than the frame and disposed between the frame and the skirt portion, which is at least located above the nose portion and extends toward the skirt portion.

In this way, the soft supporting member can separate at least part of the frame from the skirt portion so that the frame is less likely to press against the user's face under the action of an external force.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a perspective view of a conventional goggle structure;

FIG. 2 is a cross-sectional perspective view of the conventional goggle structure;

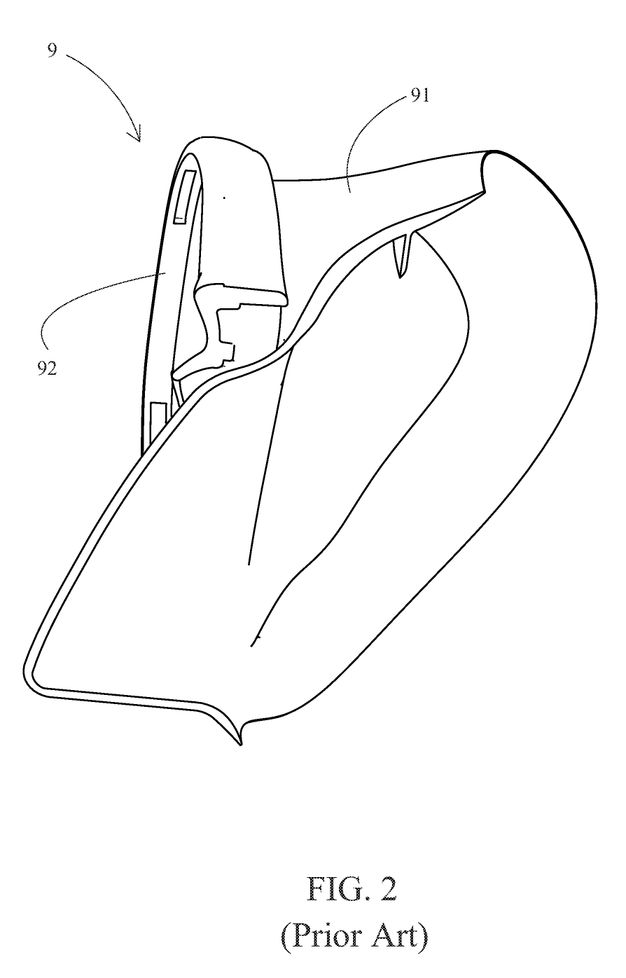

FIG. 3 is a perspective view of a goggle structure according to the first preferred embodiment of the present invention;

FIG. 4 is a cross-sectional perspective view of the goggle structure shown in FIG. 3, with the lens assembly omitted;

FIG. 5 is a top view of the goggle structure shown in FIG. 3, with the facial mask and the lens assembly omitted;

FIG. 6 is a cross-sectional plan view of the goggle structure shown in FIG. 3;

FIG. 7 is a perspective view of a goggle structure according to the second preferred embodiment of the present invention, with the facial mask and the lens assembly omitted;

FIG. 8 is a top view of the goggle structure shown in FIG. 7, with the facial mask and the lens assembly omitted; and

FIG. 9 is a cross-sectional plan view of the goggle structure shown in FIG. 7.

### DESCRIPTION OF THE PREFERRED EMBODIMENT

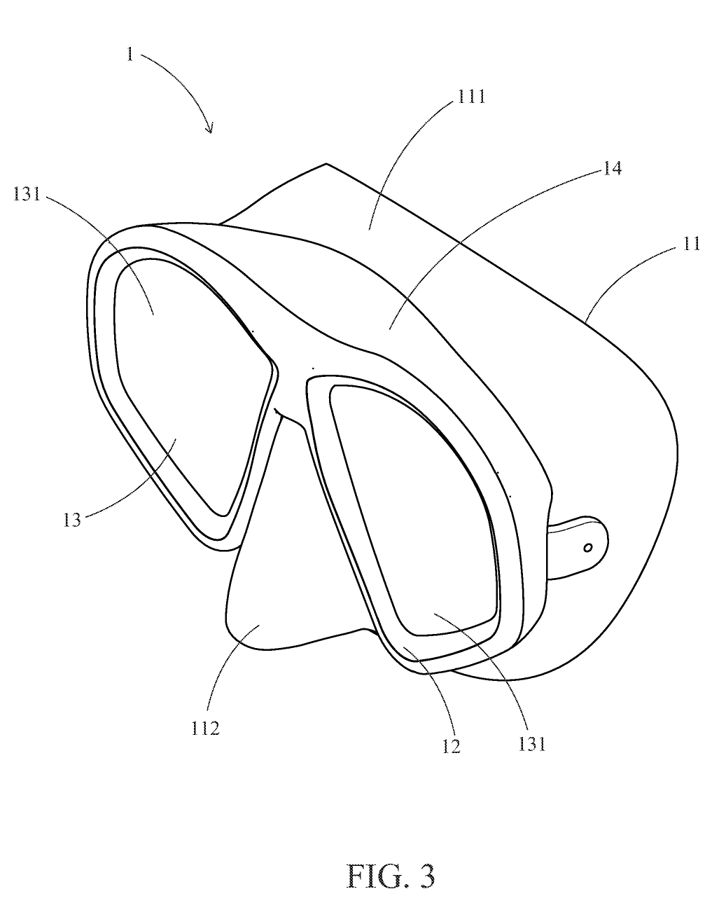

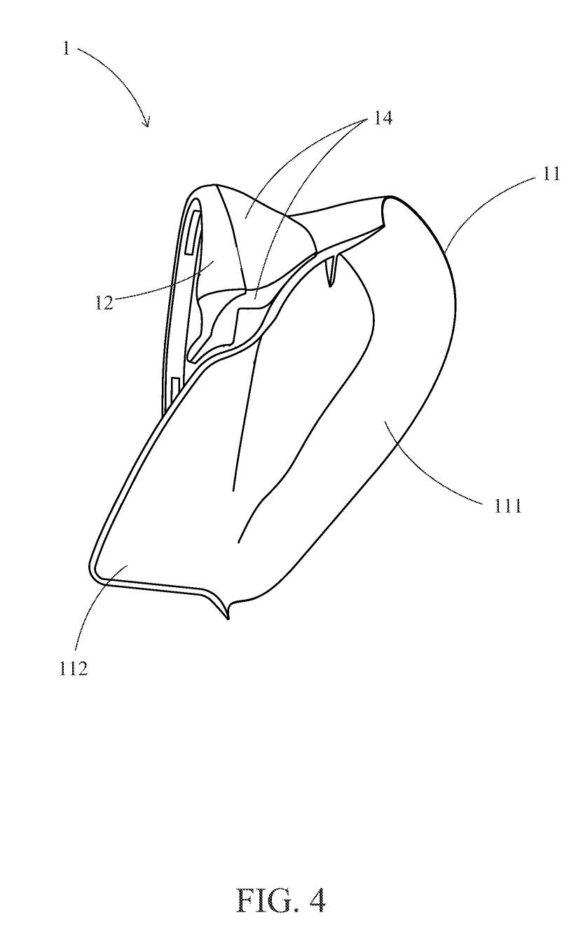

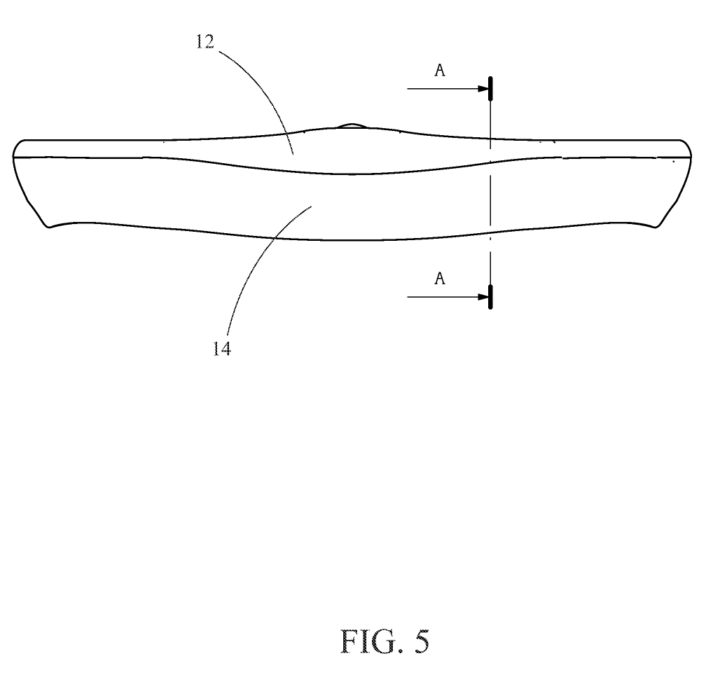

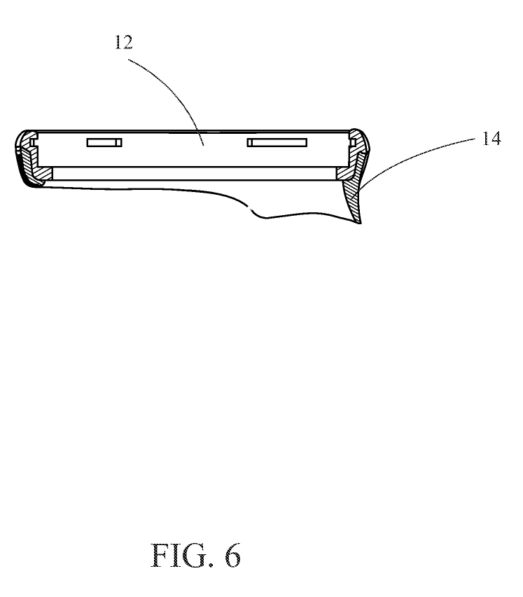

FIGS. 3 and 4 illustrate a perspective view and across-sectional perspective view of a goggle structure according to the first preferred embodiment of the present invention respectively. FIGS. 5 and 6 illustrate a top view and across-sectional plan view of the goggle structure shown in FIG. 3 (with the facial mask and the lens assembly omitted) respectively.

The goggle structure 1 of the first embodiment comprises a facial mask 11, a frame 12, a lens assembly 13 and a soft supporting member 14, which will be described in sequence hereinbelow.

The facial mask 11 may be made of a soft material, and has a skirt portion 111 and a nose portion 112. The skirt portion 111 may be formed of a sheet structure and has a shape matching the profile of a human face; thus, when the goggle structure 1 is worn by a user, the skirt portion 111 is adapted to be seamlessly attached to the user's face so that it is difficult for water to pass between the skirt portion 111 and the user's face. The nose portion 112 may also be formed of a sheet structure and have a shape matching the profile of a human nose; thus, when the goggle structure 1 is worn by the user, the nose can be accommodated in the nose portion 112.

The frame 12 may be formed of a rod that is bent continuously, and may form at least one enclosed space. The frame 12 may be made of a material selected from the following: polycarbonate (PC), polyoxymethylene (POM), acrylonitrile butadiene styrene (ABS), nylon or polypropylene (PP). The frame 12 is connected with the skirt portion 111, and may be located at the front side of the skirt portion 111. The lens assembly 13 is disposed within the frame 12, and may have at least one lens 131 (two in this embodiment).

The soft supporting member 14 is softer than the frame 12; i.e., the soft supporting member 14 has a low hardness, which may range from Shore A10 to Shore A95. The soft supporting member 14 is made of a material selected from thermoplastic rubber (TPR), thermoplastic polyurethane (TPU), thermoplastic elastomer (TPE), rubber, polyvinyl chloride (PVC) or silicone rubber.

The soft supporting member 14 is disposed between the frame 12 and the skirt portion 111 and extends towards the skirt portion (i.e., extends backwards); furthermore, the soft supporting member 14 is at least located above the nose portion 112. In this way, the soft supporting member 14 can separate part of the frame 12 located above the nose portion 112 from the skirt portion 111.

Thus, when the goggle structure 1 of this embodiment is worn by the user, the soft supporting member 14 is exactly located in front of the forehead of the user's face because the forehead is located directly above the nose portion 112. There is still be a possibility that the facial mask 11 of the goggle structure 1 can collapse when it is pressed by a force from the user or by the water pressure; however, the supporting effect provided by the soft supporting member 14 to part of the frame 12 can inhibit the part of the frame 12 located above the nose portion 112 from being pressed against the user's forehead, so the user will feel less uncomfortable.

Additionally, the soft supporting member 14 may further extend to the left side and right side of the lens assembly 13 respectively to separate part of the frame 12 located at both the left and right sides of the lens assembly 13 from the skirt portion 111. Then, the part of the frame 12 located at the left side and the right side of the lens assembly 13 will not press against the user's face even when the facial mask 11 of the goggle structure 1 collapses. As can be known from the above description, the soft supporting member 14 is not merely limited to be located above the nose portion 112.

To further enhance the supporting effect of the soft supporting member 14, the soft supporting member 14 may be fixed with the frame 12; and the soft supporting member 14 may come into contact with the skirt portion 111 when extending towards the skirt portion 111. The soft supporting member 14 and the frame 12 may be fixed together through overmolding in such a way that a specific part of the frame 12 is covered by the soft supporting member 14. Through overmolding, a large joining force can be obtained between the soft supporting member 14 and the frame 12.

Apart from the overmolding process, the soft supporting member 14 may also be fixed to the frame 12 through adhesion. In detail, the soft supporting member 14 may be separately fabricated, and then adhered to a specific location on the frame 12 by means of an adhesive (not shown), thus achieving the purpose of fixing the soft supporting member 14 with the frame 12.

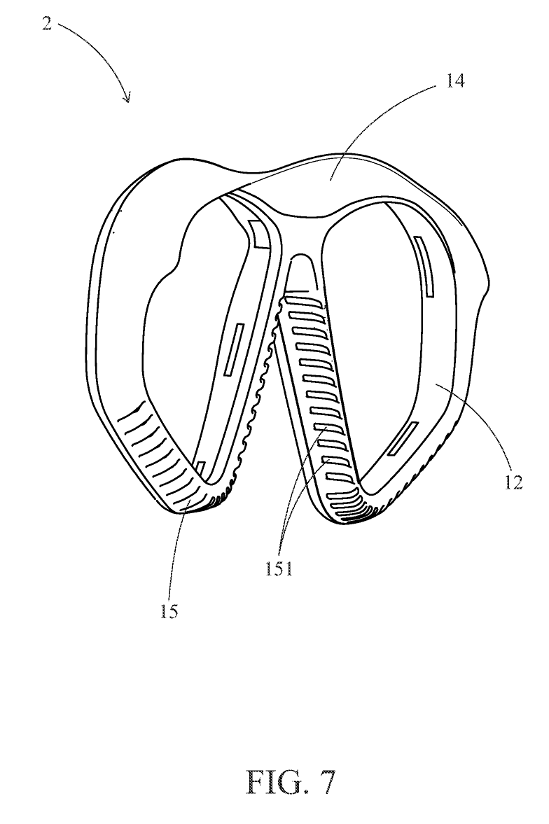

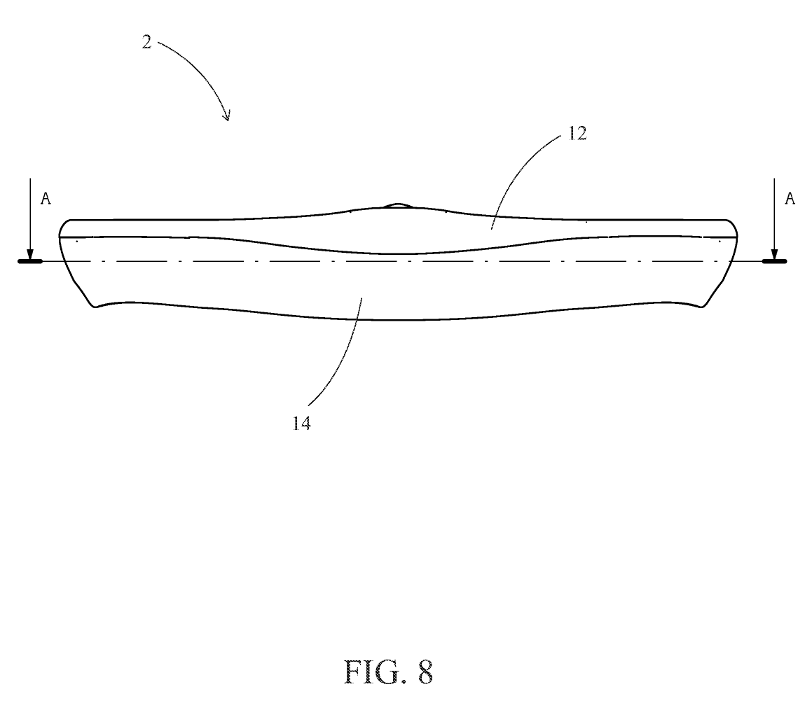

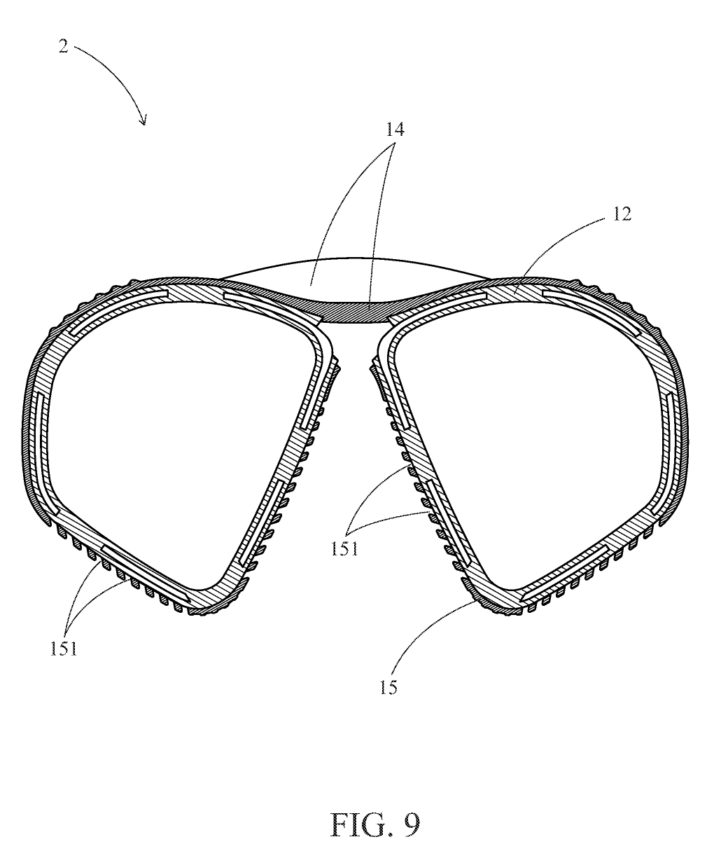

In reference to FIGS. 7 through 9, a perspective view, a top view and a cross-sectional plan view of a goggle structure according to the second preferred embodiment of the present invention are shown therein respectively (with the facial mask and the lens assembly omitted).

The goggle structure 2 of the second preferred embodiment differs from the goggle structure 1 of the first preferred embodiment in that the goggle structure 2 further comprises two soft cushioning members 15. This will be detailed as follows.

The two soft cushioning members 15 are softer than the frame 12; i.e., the soft cushioning members 15 have a low hardness which may range from Shore A10 to Shore A95. The soft cushioning members 15 may be made of a material identical to that of the soft supporting member 14; i.e., the soft cushioning members 15 may be made of a material selected from thermoplastic rubber (TPR), thermoplastic polyurethane (TPU), thermoplastic elastomer (TPE), rubber, polyvinyl chloride (PVC) or silicone rubber.

The two soft cushioning members 15 are connected with the frame 12 and located at the left and right sides of the nose portion (reference may be made to the nose portion 111 shown in FIG. 3) of the facial mask respectively. The connection between the soft cushioning members 15 and the frame 12 may also be achieved through overmolding or adhesion.

Additionally, the two soft cushioning members 15 may be integrally connected with the soft supporting member 14, and the two soft cushioning members 15 may also be integrally connected with each other; in other words, the goggle structure 2 may be described as comprising a single soft cushioning member 15, two sides of which are located at the left side and the right side of the nose portion respectively.

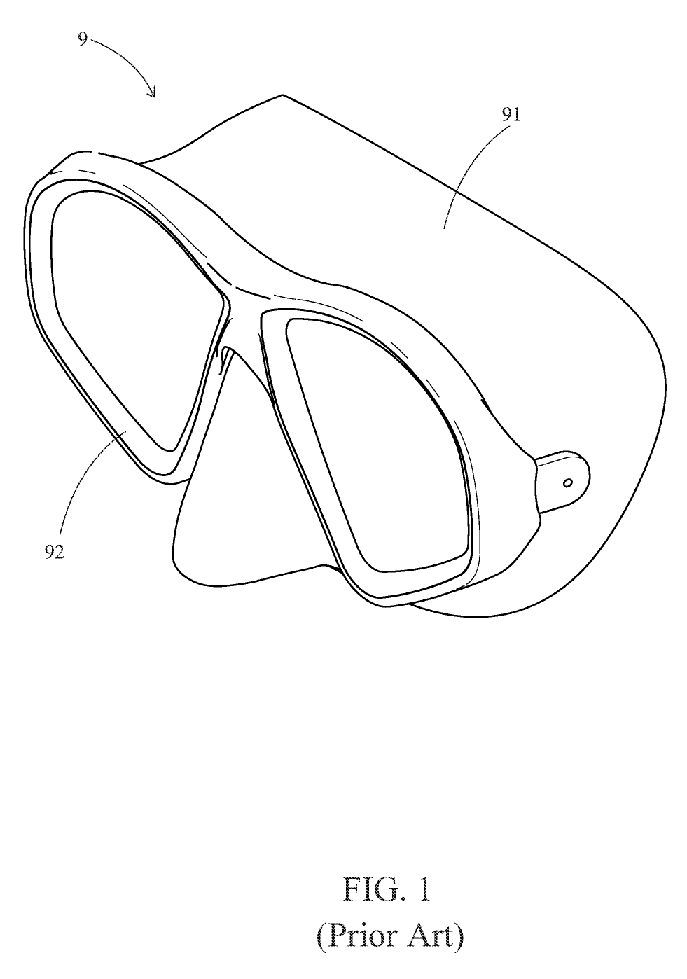

When the goggle structure 2 is worn by the user, the two soft cushioning members 15 are located at the left side and the right side of the user's nose respectively. In case the user's nose is larger than the nose portion, the user's nose will completely fill the nose portion and compress the two soft cushioning members 15. Because of the low hardness of the soft cushioning members 15, the user will not feel uncomfortable even when the nose compresses the soft cushioning members 15. In contrast, with the conventional goggle structure that has a hard frame as shown in FIG. 1, the user would feel uncomfortable when the nose compresses the hard frame.

Furthermore, the two soft cushioning members 15 may each have a plurality of grooves 151 spaced apart from each other. Through arrangement of the grooves 151, a large contact area can be achieved between the user's hand and the soft cushioning members 15 when the user handles the goggle structure 2 with the hand. Thereby, a large frictional force is obtained to facilitate the handling of the goggle structure 2.

It shall be appreciated that in other embodiments (not shown), the two soft cushioning members 15 may be detachable from the soft supporting member 14; i.e., it is unnecessary for the two soft cushioning members 15 to be integrally connected with the soft supporting member 14. Additionally, the two soft cushioning members 15 may also be detached from each other instead of being integrally connected. In other words, the frame 12 of the goggle structure may have a plurality of discrete soft components.

According to the above descriptions, the goggle structure of the present invention at least has the following features:

1. the soft supporting member can provide additional support for the frame, so at least part of the frame will not come into contact with the user's face;

2. the soft supporting member may be overmolded with the frame to reduce the difficulty in manufacturing and assembling the goggle structure;

3. the soft supporting member may be separately fabricated and then fixedly adhered to the frame;

4. the soft cushioning members can prevent the user's nose from compressing the frame, so the user will feel less uncomfortable; and

5. the soft cushioning members can provide an anti-slip effect, so the user can handle the goggle structure easily.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A goggle structure, comprising:
a facial mask, having a skirt portion and a nose portion connected with the skirt portion;
a frame, connected to the skirt portion;
a lens assembly, disposed within the frame; and
a soft supporting member, softer than the frame and disposed between the frame and the skirt portion, and at least located above the nose portion, the soft supporting member extending toward the skirt portion.

2. The goggle structure of claim 1, wherein the soft supporting member extends toward the skirt portion, and is in contact with the skirt portion.

3. The goggle structure of claim 1, wherein the soft supporting member is fixed with the frame.

4. The goggle structure of claim 3, wherein the soft supporting member is adhered to the frame.

5. The goggle structure of claim 1, further comprising at least one soft cushioning member which is softer than the frame and connected with the frame, wherein both sides of the soft cushioning member is located at a left side and a right side of the nose portion respectively.

6. The goggle structure of claim 1, further comprising two soft cushioning members which are softer than the frame and connected with the frame, wherein the two soft cushioning members are located at a left side and a right side of the nose portion, respectively.

7. The goggle structure of claim 6, wherein each of the two soft cushioning members has a plurality of spaced apart recesses.

8. The goggle structure of claim 6, wherein the two soft cushioning members are integrally connected with the soft supporting member.

9. The goggle structure of claim 6, wherein the soft cushioning member is made of a material selected from thermoplastic rubber, thermoplastic polyurethane, thermoplastic elastomer, polyvinyl chloride, rubber or silicone rubber.

10. The goggle structure of claim 1, wherein the soft supporting member is made of a material selected from thermoplastic rubber, thermoplastic polyurethane, thermoplastic elastomer, polyvinyl chloride, rubber or silicone rubber.

11. The goggle structure of claim 1, wherein the frame is made of a material selected from polycarbonate, polyoxymethylene, acrylonitrile butadiene styrene, nylon or polypropylene.
